(19) United States
(12) Patent Application Publication
(10) Pub. No.: US 2003/0138499 A1
(43) Pub. Date: Jul. 24, 2003

(54) Stable surgical irrigating solutions

(76) Inventors: Mandar V. Shah, Arlington, TX (US); Uday Doshi, Fort Worth, TX (US); John C. Liao, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/340,431

(22) Filed: Jan. 10, 2003

Related U.S. Application Data
(63) Continuation of application No. 09/830,657, filed on Apr. 30, 2001, now abandoned.
Application No. 09/830,657 is a 371 of international application No. PCT/US99/28417, filed on Dec. 1, 1999.
(60) Provisional application No. 60/112,664, filed on Dec. 17, 1998.

(51) Int. Cl. A61K 31/70; A61K 31/375; A61K 31/353; A61K 31/343; A61K 33/06; A61K 33/00; A61K 33/42

(52) U.S. Cl. 424/682; 424/717; 514/23; 514/159; 514/456; 514/454; 514/469; 514/474; 424/601

(57) ABSTRACT

Improved stability surgical irrigating solutions and methods of use are disclosed. The compositions comprise bifunctional compounds (i.e., compounds comprising antioxidant and antiinflammatory moieties) and physiological antioxidants to stabilize the bifunctional compounds. The compounds are useful in preventing and treating inflammatory and other disorders incident to surgery.

### BACKGROUND OF THE INVENTION

The present invention is directed to the provision of improved stability surgical irrigating solutions comprising particular bifunctional compounds. More specifically, the present invention is directed to stable surgical irrigating solutions containing a bifunctional compound (i.e., a compound comprised of antioxidant and anti-inflammatory moieties) and an amount of physiological antioxidant (e.g., ascorbate) to stabilize the bifunctional compound. The present invention is also directed to various methods of using the compositions of the present invention, including the treatment of ocular inflammation associated with ophthalmic disease and ophthalmic surgery.

Ocular surgery can result in various post-surgical complications to the eye. Such complications may include: 1) loss of vascular blood barrier function; 2) neutrophil accumulation; 3) tissue edema including conjunctiva swelling, conjuctiva congestion and corneal haze; 4) cataract formation; 5) cellular proliferative disorders including neovascularizations, fibrosis and posterior capsule opacification; and 6) loss of membrane integrity including decrease in docosahexanenoic acid levels in membrane phospholipids. Many of these complications are further potentiated in diabetic patients who are at risk for many ocular pathologies.

Refractive surgery typically involves the modification of the cornea in myopic patients to correct the focus of light on the retina. Examples of such surgeries include radial keratotomy (radial slices in the cornea), photorefractive keratotomy (laser ablation of the epitheilial and stromal layers of the cornea), LASIK (slicing a cornea flap and removing part of the stromal layer followed by the replacement of the flap), as well as procedures involving the insertion of corneal rings or phakic intraocular lens (“IOL”). During these and other corneal surgeries the cornea is typically bathed with a surgical irrigating solution. Due to the traumatic insult of such procedures, however, various inflammatory events or other tissue or cellular complications may arise.

Cataract surgery involves the removal of the cataractous lens and replacement with an IOL. In such surgeries the entire lens is removed in one piece, or the lens is broken down into smaller pieces and suctioned out of the lens capsule by phacoemulsification techniques. In some cases following surgery, however, opacification of the posterior capsule forms, inhibiting clear vision and potentially necessitating further surgery.

Posterior segment surgery, due to the severity of the surgical procedure, can cause extensive tissue damage at both the acute and chronic phases of the recovery process. The acute phase of the postsurgical period is characterized by both ocular neovascularization and tissue edema. This is caused by breakdown of the blood aqueous and blood retinal barrier functions resulting in sustained vascular permeability following the surgical trauma. The presence of elevated inflammatory and serum factors induce cell proliferation during the normal wound healing process. Slitlamp clinical examinations at 24 hours have indicated extensive anterior chamber flare and cell influx, conjunctiva congestion and swelling (with discharge), iritis, and corneal haze. See for example, Kreiger, A. E., Wound Complications In Pars Plana Vitrectomy, Retina, volume 13, No. 4, pages 335-344 (1993); Cherfan, G. M., et al., Nuclear Sclerotic Cataract After Vitrectomy for Idiopathic Epiretinal Membranes Causing Macular Pucker, American Journal Of Ophthalmology, volume 111, pages 434-438 (1991); Thompson, J. T., et al., Progression of Nuclear Sclerosis and Long-term Visual Results of Virectomy With Transforming Growth Factor Beta-2 for Macular Holes, American Journal Of Ophthalmology, volume 119, pages 48-54 (1995) and Dobbs, R. E., et al., Evaluation Of Lens Changes In Idiopathic Epiretinal Membrane, volume 5, Nos. 1 & 2, pages 143-148 (1988).

The chronic phase of the postsurgical period is characterized by more severe complications that can necessitate additional surgery. These include an incidence of recurrent retinal detachment, epiretinal proliferation, neovascular glaucoma, corneal problems, vitreous hemorrhage, rate of cystoid macular edema, and occurrence of cataract formation within six months of surgery.

Neurosurgery is another important area where there is a need for irrigating solutions which better stabilize irreplaceable tissue. It is well known that destroyed nerve cells, for the most part, are not regenerated.

Currently, surgical irrigating solutions employed during the surgeries described above, like those described in U.S. Pat. No. 4,550,022 (Garabedian et al.), typically contain sodium, potassium, magnesium, calcium, chloride, and bicarbonate ions as well as dextrose and glutathione in proportions consistent with the osmotic stability and continued metabolism of the tissue cells. These irrigating solutions are generally prepared by mixing a first solution which provides the bicarbonate and a second solution which provides the calcium, magnesium, dextrose and glutathione. The first and second solutions are generally stored as for extended periods of time and mixed within 24 hours of use.

Such irrigating solutions, however, do not contain additional therapeutic compounds which may aid in the prevention or amelioration of inflammation or other tissue or cellular trauma resulting from surgery.

U.S. Pat. Nos. 5,607,966 and 5,811,438, issued to Hellberg et al., disclose, inter alia, improved surgical irrigating solutions comprising bifunctional compounds having cytoprotective efficacy. The bifunctional compounds of the Hellberg et al. patents, however, are inherently unstable over time in pre-mixed component solutions of the irrigating solutions. Those compositions, therefore, possess a limited shelf-life due to their instability. The present invention improves on such compositions by improving the shelf-life stability of the pre-mixed component solutions of the compositions.

### SUMMARY OF INVENTION

The present invention is directed to improved two-part surgical irrigating solution systems. The improved irrigating solutions comprise particular bifunctional compounds (i.e., compounds comprised of antioxidant and anti-inflammatory moieties) and physiological antioxidants as a stabilizers of the bifunctional compounds.

Due to the inherent sensitivity of these bifunctional compounds to oxidation, the pre-mixed component solutions of previous surgical irrigating solutions containing the bifunctional compounds have possessed limited shelf-life. The compositions of the present invention have been formulated to stabilize the bifunctional compounds. It has been found that the addition of physiological antioxidants to the pre-mixed component solutions stabilizes the bifunctional compounds while not interfering with their cytoprotective efficacy and, hence, the cytoprotective efficacy of the surgical irrigating compositions.

The compositions of the present invention are useful in surgical applications. The compositions are particularly well suited for ophthalmic surgery. As stated above, the bifunctional compounds exhibit cytoprotective effects. These compounds include both a non-steroidal anti-inflammatory agent (“NSAIA”) moiety and an antioxidant moiety. The bifunctional compounds of the present invention are capable of protecting against cellular damage by a wide range of insults. Since the compounds provide this protection by decreasing free radical or oxidative damage, reducing enzyme mediated inflammation, and improving site delivery, this therapy represents an improved two-pronged approach to the prevention or amelioration of inflammatory events coincident with surgical manipulations.

The compositions of the present invention may also be useful in the irrigation of neural tissue and other sensitive tissues during surgery.

### DETAILED DESCRIPTION OF INVENTION

The present invention is directed to improved two-part surgical irrigating solution systems. The two parts comprise a buffered, neutral solution and an acidic solution. The compositions of the two solutions are individually stable and may be separately stored for long periods. When mixed together, the two solutions form a tissue irrigating solution that may be used for surgery during the next 24 hours. Preferably, however, the compositions will be used within 6 hours of mixing. The mixed solutions are particularly useful for ocular surgery. The compositions of the present invention contain constituents which serve not only as a physiological buffer but also as a metabolic energy source required for cell viability and maintenance of normal cellular/tissue functions including, but not limited to, the maintenance of normal physiological functions of the eye, such as cornea and lens transparency, endothelial cell integrity and retinal function. The irrigating solutions also contain therapeutically effective amounts of bifunctional compounds to reduce or ameliorate inflammatory and other tissue or cytotoxic events, which may occur during ocular surgery. The compositions are also useful in maintaining the stability of other sensitive tissues, including, but not limited to, neural tissue during neurosurgery.

The combined irrigating solutions contain the necessary ions for tissue/cellular stability (Ca++, Mg++, Na+, K+ and Cl−,) in a buffering system, as well as dextrose, one or more physiological antioxidants as stabilizing agents, and a therapeutically effective amount of one or more cytoprotective bifunctional compounds of formula (I):

A-X—(CH₂)ₙ—Y—(CH₂)ₘ—Z (I)

wherein:

A is an non-steroidal anti-inflammatory agent (NSAIA) originally having a carboxylic acid;

A-X is an ester or amide linkage derived from the carboxylic acid moiety of the NSAIA, wherein X is O or NR;

R is H, C₁-C₆ alkyl or C₃-C₆ cycloalkyl;

Y, if present, is O, NR, C(R)₂, CH(OH) or S(O)_{n′};

n is 2 to 4 and m is 1 to 4 when Y is O, NR, or S(O)_{n′};

n is 0 to 4 and m is 0 to 4 when Y is C(R)₂ or is not present;

n is 1 to 4 and m is 0 to 4 when Y is CH(OH);

n′ is 0 to 2; and

Z is:

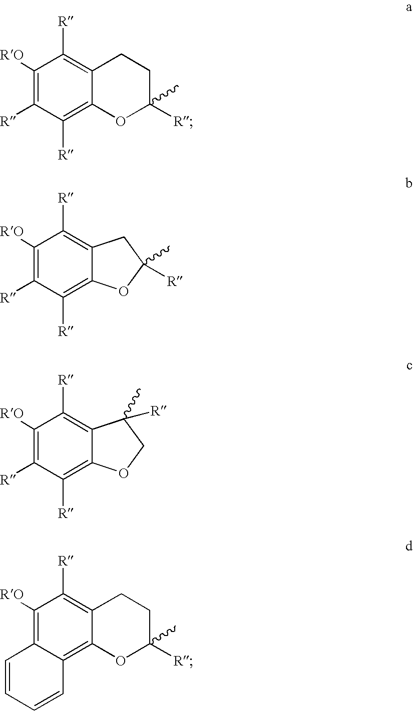

wherein:

R′ is H, C(O)R, C(O)N(R)₂, PO₃⁻, or SO₃⁻; and

R″ is H or C₁-C₆ alkyl.

The bifunctional compounds of the present invention also include various stereoisomers or racemic mixtures of any of the compounds contemplated within formula (I), and pharmaceutically acceptable salts of the compounds of formula (I).

The bifunctional compounds of the present invention contain a non-steroidal: anti-inflammatory agent, “A”, originally having a carboxylic acid moiety. A number of chemical classes of non-steroidal anti-inflammatory agents have been identified. The following text, the entire contents of which are incorporated herein by reference to the extent it refers to NSAIAs having a carboxylic acid, may be referred to for various NSAIA chemical classes: CRC Handbook of Eicosanoids: Prostaglandins, and Related Lipids, Volume II, Drugs Acting Via the Eicosanoids, pages 59-133, CRC Press, Boca Raton, Fla. (1989). The NSAIA may be selected, therefore, from a variety of chemical classes including, but not limited to, fenamic acids, such as flufenamic acid, niflumic acid and mefenamic acid; indoles, such as indomethacin, sulindac and tolmetin; phenylalkanoic acids, such as suprofen, ketorolac, flurbiprofen, ibuprofen and diclofenac. Further examples of NSAIAs are listed below:

| | | |
|---|---|---|
| loxoprofen | tolfenamic acid | indoprofen |
| pirprofen | clidanac | fenoprofen |
| naproxen | fenclorac | meclofenamate |
| benoxaprofen | carprofen | isofezolac |
| aceloferac | fenbufen | etodolic acid |
| fleclozic acid | amfenac | efenamic acid |
| bromfenac | ketoprofen | fenclofenac |
| alcofenac | orpanoxin | zomopirac |
| diflunisal | pranoprofen | zaltoprofen |

The preferred compounds of formula (I) are those wherein “A” is selected from the ester or amide derivatives of naproxen, flurbiprofen or diclofenac. The most preferred compounds are those wherein “A” is selected from the ester or amide derivatives of naproxen or flurbiprofen.

With respect to the other substituents of the compounds of formula (I), the preferred compounds are those wherein:

X is O or NR;

R is H or C₁ alkyl;

Y is CH(OH), and m is 0 to 2 and n is 1 or 2, or Y is not present, and m is 1 or 2 and n is 0 to 4;

Z is a, b or d;

R′ is H or C(O)CH₃; and

R″ is CH₃.

The most preferred compounds are those wherein:

X is O or NR;

R is H;

Y is not present;

m is 0 or 1;

n is 1;

Z is a, or b;

R′ is H; C(O)CH₃; and

R″ is CH₃.

The following compounds are particularly preferred:

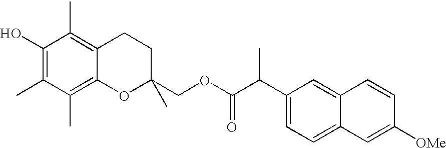

2-(6-hydroxy-2,5,7,8-tetramethyl-3,4-dihydro-2H-benzo[1,2-b]pyran-2-yl)methyl 2-(6-methoxy-2-naphthyl)propionate (“Compound A”);

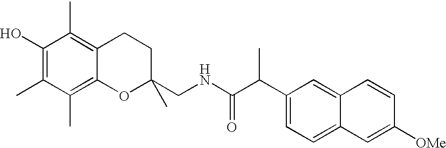

N-(2-(6-hydroxy-2,5,7,8-tetramethyl-3,4-dihydro-2H-benzo[1,2-b]pyran-2-yl)methyl) 2-(6-methoxy-2-naphthyl)propionamide (“Compound B”);

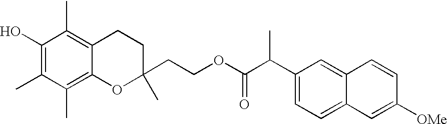

2-(6-hydroxy-2,5,7,8-tetramethyl-3,4-dihydro-2H-benzo[1,2-b]pyran-2-yl)ethyl 2-(6-methoxy-2-naphthyl)propionate (“Compound C”);

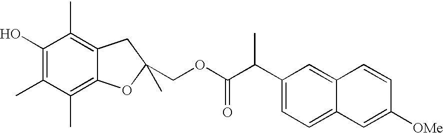

2-(5-hydroxy-2,4,6,7-tetramethyl-2,3-dihydro-benzo[1,2-b]furan-2-yl)methyl 2-(6-methoxy-2-naphthyl)propionate (“Compound D”);

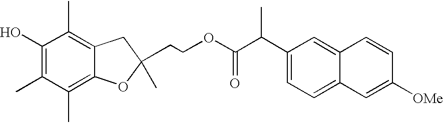

2-(5-hydroxy-2,4,6,7-tetramethyl-2,3-dihydro-benzo[1,2-b]furan-2-yl)ethyl 2-(6-methoxy-2-naphthyl)propionate (“Compound E”); and

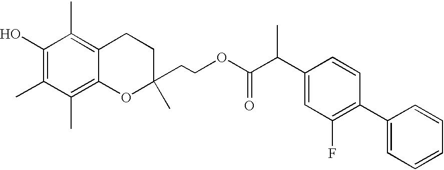

2-(6-hydroxy-2,5,7,8-tetramethyl-2,3-dihydro-2H-benzo[1,2-b]pyran-2-yl)ethyl 2-(3-fluoro-4-phenyl-phenyl)propionate (“Compound F”).

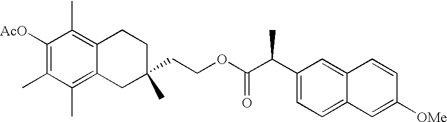

(S)-6-methoxy-α-methyl-naphthaleneacetic acid, (R)-2-(6-acetoxy-3,4-dihydro-2,5,7,8-tetramethyl-2H-1-benzopyran-2-yl)ethyl ester (“Compound G”),

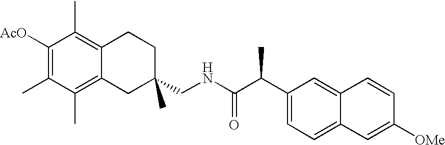

(R)N-(2-(6-acetoxy-2,5,7,8-tetramethyl-3,4-dihydro-2H-benzo[1,2-b]pyran-2-yl)methyl) (S)(2-(6-methoxy-2-naphthyl)propionamide (“Compound H”);

The most preferred bifunctional compound of the present invention is:

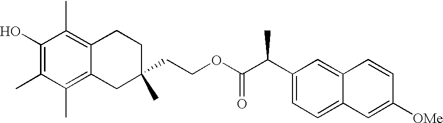

(S)-6-methoxy-α-methyl-naphthaleneacetic acid, (R)-2-(3,4-dihydro-6-hydroxy-2,5,7,8-tetramethyl-2H-1-benzopyran-2-yl)ethyl ester (“Compound X”), which is a particular stereoisomer of Compound C.

The compounds of formula (I) possess antinflammatory, antioxidant and antiproliferative activity. The compounds of formula (I) may be prepared by methods disclosed in U.S. Pat. No. 5,607,966 (Hellberg et al.), the entire contents of which are incorporated herein by reference.

The irrigating compositions of the present invention will contain one or more compounds of formula (I) useful for the prevention or amelioration of various types of cellular damage incident to surgical procedures. In particular, these compositions may be used for the prevention or amelioration of inflammation where prostaglandins, leukotrienes, cytokines and other proinflammatory agents are known to participate.

As stated above, ocular surgeries, such as refractive, cataract, posterior-segment and glaucoma filtration surgeries, can result in various post-surgical complications to the eye. Such complications may include, but not limited to, loss of vascular blood barrier function, tissue edema including conjunctiva swelling and congestion, corneal haze, cataract formation, retinal detachment, epiretinal proliferation, neovascular glaucoma, posterior capsule opacification, vitreous hemorrhage, and cystoid macular edema neovascularizations. The frequency of these and other complications may be lessened by facilitating the prevention or amelioration of inflammation or other cellular or tissue discorders including cellular proliferative responses, by employing the improved irrigating solutions of the present invention during surgery.

The concentrations of the compounds of formula (I) in the surgical irrigating solutions will depend on various factors, including the nature and severity of the condition to be treated. The irrigating solutions of the present invention, however, will contain a compound of formula (I) in a therapeutically effective amount. As used herein, a “therapeutically effective amount” is that amount of a compound of formula (I) which prevents, reduces or ameliorates inflammation or other cellular or tissue trauma. In general, however, the irrigating solutions will contain one or more compounds of formula (I) in a final concentration of about 0.01 to 100 μmoles/L (“μM”). Preferred irrigating solutions will contain one of more compounds of formula (I) in a final concentration of about 0.2 to 5 μM, which generally corresponds to approximately 0.00001 to 0.00025 percent weight/volume (“% w/v”).

As stated above, the irrigating solutions of the present invention also contain electrolytes, a bicarbonate buffering system, dextrose and one or more physiological antioxidants as stabilizing agents for the formula (I) compounds. Optionally, the compositions of the present invention will also contain one or more solubilizing agents.

The electrolytes are provided in proportions conducive to cellular integrity and continued cell metabolism. Preferred, final irrigating solutions will contain from about 130 to about 180 millimoles/L (“mM”) Na+, from about 3 to about 10 mM K+, from about 1 to about 5 mM Ca++, from about 0.5 to about 4 mM Mg++ and from about 130 to about 210 mM Cl−. To maintain osmotic stability of the cells, the osmolality of the irrigation solutions will be between about 250 and about 350 mOsm/kg and preferably about 290-320 mOsm/kg. So as to closely match the physiological pH of 7.4, the pH of the final irrigating solution will be between about 6.8 and about 8.0 and preferably about 7.2-7.8. To maintain the fluid pump system, the bicarbonate concentration in the final irrigating solution will be between about 10 and about 50 mM. To stabilize the pH, an additional buffering agent is provided, such as phosphate or citrate. Preferably the buffering agent is phosphate which is provided in sufficient quantity so that the final phosphate concentration of the irrigating solution is between about 1 and about 5 mM. The final irrigating solution will also contain between about 2 and about 10 mM dextrose.

The preferred, neutral component solution provides the phosphate and bicarbonate buffering moieties, preferably in the form of dibasic sodium phosphate and sodium bicarbonate. The pH of the neutral solution is adjusted to a physiological pH, preferably between about 7.2 and 7.8, and most preferably, 7.4. The pH of a bicarbonate-containing solution if preferably above about 8.0 to prevent decomposition of the bicarbonate. It has been found, however, that the bicarbonate may be stabilized if it is added to a solution with a pH of above about 8 and thereafter adjusted to a pH between 7 and 8. Accordingly, when the preferred neutral solution is prepared, Na₂HPO₄ is added prior to the addition of NaHCO₃ so that NaHCO₃ is dissolved in a solution with a pH of between about 8 and about 9. The solution is thereafter adjusted with dilute acid, such as H₂SO₄, H₃PO₄ or HCl, to the desired final pH of the neutral solution. Alternatively, carbon dioxide may be added to adjust the pH.

Potassium and additional sodium ions are provided in the irrigating solutions in the form of sodium and potassium salts, such as sodium or potassium chlorides, sulfates, acetates, citrates, lactates, and gluconates. The sodium and potassium ions will be compatible with all of the moieties present in the finished tissue irrigating solution, and sodium chloride and potassium chloride may be added to either component solution or divided between the solutions. However, in view of the fact that the neutral solution provides the buffer system, the pH of the final irrigation solution may be more accurately determined if all compatible salts are included in the neutral solution.

The acidic solution provides the Ca++ ion in the form of calcium chloride, the Mg++ ion in the form of magnesium chloride, dextrose, one or more bifunctional compounds of formula (I) and one or more physiological antioxidants to stabilize the bifunctional compounds. The pH of the acidic solution is adjusted between about 4-7, preferably between 4.5-6 and most preferably at 5, to provide long-term stability of dextrose and enhance the stability of the bifunctional compounds of formula (I).

The physiological antioxidants may be selected from antioxidants which are endogenously present in a mammal, provide for the stabilization of compounds of formula (I) in an acidic composition, do not cause substantial discoloration or precipitates to form in the acidic or final irrigating solutions, and which do not cause adverse side effects or interfere with the activity of formula (I) compounds in vivo. Examples of such antioxidants include, but are not limited to, vitamin E, vitamin A, vitamin C (ascorbic acid or salts thereof), reduced glutathione, and derivatives thereof, or suitable combinations thereof. Other physiological antioxidants which possess a higher oxidative potential than the formula (I) compounds may be also be used as stabilizers in the compositions, provided such antioxidants conform with the above criteria. The most preferred physiological antioxidant is ascorbic acid/ascorbate.

The amount of physiological antioxidant included in the acidic solution will vary depending on various factors such as the particular compound or compounds of formula (I) to stabilize, the dilution ratio of the acidic solution in the final irrigating solution and the efficacy of the antioxidant(s). However, such an amount will be that amount which stabilizes the compounds of formula (I) in the acidic solutions. As used herein, a “stabilizing amount” or “amount to stabilize” refers to that amount of antioxidant which prevents or limits the oxidation and/or breakdown of a compound of formula (I) in the acidic solution. Preferred antioxidant amounts are about 0.005 to 0.5% (w/v) in the acidic solutions. The antioxidants may also provide some stabilization of formula (I) compounds in the final surgical irrigation solution.

As stated above, ascorbic acid/ascorbate is the most preferred physiological antioxidant. Ascorbate ions may be added to the acidic solution in the form of ascorbic acid and/or a soluble salt of ascorbate including, but not limited to, sodium ascorbate or calcium ascorbate. In general, the ascorbate ion concentration of the acidic solution will be about 0.5 to 5 mM which generally corresponds to about to 0.01-to 0.1% (w/v). The preferred ascorbate source in the acidic solution will be a combination of ascorbic acid and sodium ascorbate. The preferred amount of ascorbate ion in the acidic solution will be about 0.5 to 3.1 mM, which can be sourced by using a combination of about 0.0008 to 0.005% (w/v) of ascorbic acid and about 0.009 to 0.058% (w/v) of sodium ascorbate. The most preferred acidic solutions of the present invention will contain about 0.023% (w/v) of sodium ascorbate and about 0.002% (w/v) of ascorbic acid.

Optionally, an amount of acetate ion (e.g., sodium acetate) of about 0.25-1.0% (w/v) may be combined with the antioxidants to aid in the stabilization of formula (I) compounds. The addition of acetate to the acidic solutions, however, may necessitate buffering agent adjustments in the neutral solution to compensate for the pH effects of the additional acetate ions on the combined irrigating solutions.

One or more solubilizing agents may also be added to the acidic solutions of the present invention to solubilize a compound of formula (I). Typical solubilizing agents include polysorbate 20, 40, 60 and 80; Pluronic® F-68, F-84 and P-103 (BASF Corp., Parsippany, N.J.); cyclodextrin; tocopherol polyethyleneglycol succinate (TPGS); polyoxyl 35 castor oil (Cremephor EL®); polyoxyl hydrogenated castor oil (RH-40®); polyethylene glycol 660 hydroxysterate (SOLUTOL® HS15), as well as other agents known in the art. Cremephor EL®, RH-40′, and SOLUTOL® HS15 are available from BASF, Corp. The most preferred solubilizing agent is polyoxyl-35 castor oil. The amount of solubilizing agent included in the acidic compositions will vary, depending on the particular acidic formulation and, in particular, the compound or compounds of formula (I) contained in the acidic solution. However, the amount of solubilizing agent to be added to the acidic solutions will be an amount that solubilizes or partially solubilizes the compounds of formula (1). In general, such an amount will be about 0.5 to 10.0% (w/v). Preferred amounts are about 0.5 to 5.0% (w/v). Preferred amounts of polyoxyl 35-castor oil are about 0.5 to 2.0% (w/v). The most preferred amount of polyoxyl 35-castor oil is 1.25% (w/v).

Due to the acidic solution having a low pH, it is preferable that the volume of the neutral solution greatly exceeds the volume of the acidic solution and that the acidic solution contains no buffering agents, or only low concentrations of mild buffering agents. Such mild buffering agents may include citrate, phosphate or acetate buffers. Ascorbate, itself, or in combination with other agents, may also provide mild buffering activity. The large volume of buffered neutral solution may be adjusted very close to the final pH of the irrigating solution and will be relatively unaffected by the addition of the small volume of the acidic solution. Preferably, the ratio of the neutral solution volume to the acidic solution volume is about 10 to 1 to about 40 to 1. The most preferred ratio is 24 to 1.

The acidic solution is preferably prepared and filled under nitrogen purge. Nitrogen purging further limits the breakdown of labile components in the solutions. Nitrogen purging of the acidic solution may be achieved by first cooling water for injection under nitrogen, compounding and addition of the various ingredients to the cooled water under a head of nitrogen, purging the resultant acidic solution with nitrogen and then filling the solution in a receptacle with a nitrogen head space. The amount and duration of purging the acidic solution with nitrogen may vary, depending on the apparatus employed and the ingredients contained in the compositions, but will preferably be efficaciously performed in a relatively short time period, such as 0.5 to 2.0 hours.

Although the description of the concentration of components included in the compositions in molar terms is useful to describe the amounts of active species present in the compositions of the present invention, it is also useful to describe the concentration of the components as percent weight/volume of their salt and/or hydrated forms. Thus, preferred, final irrigating solutions may contain about 0.63 to 0.87% w/v sodium chloride, about 0.022 to 0.075% w/v potassium chloride, about 0.01 to 0.07% w/v dibasic sodium phosphate (anhydrous), about 0.08 to 0.42% w/v sodium bicarbonate, about 0.015 to 0.073% w/v calcium chloride dihydrate, about 0.01 to 0.08% w/v magnesium chloride hexahydrate, about 0.0 to 0.008% w/v sodium citrate, about 0.04 to 0.18% w/v dextrose, about 0.00001 to 0.00025% w/v of one or more compounds of formula (I), about 0.02 to 0.4% w/v of one or more solubilizing agents, about 0.00003 to 0.0002% w/v ascorbic acid, about 0.00036 to 0.0023% w/v sodium ascorbate and minor amounts of hydrochloric acid and sodium hydroxide or other organic acids and bases useful to bring the neutral and acidic solutions to the desired pHs, respectively.

The neutral and acidic component solutions of the irrigating solutions of the present invention will be sterilized by standard techniques prior to packaging. Generally, sterilization will be carried out by autoclaving the neutral solution and sterile filtering the acidic solution, although other appropriate sterilizing techniques known to those skilled in the art may be employed. The component solutions may be stored in various containers including, but not limited to, clear or amber colored (to further inhibit oxidation of the components and, in particular, compounds of formula (I)) glass or plastic bottles, or plastic surgical bags. To avoid the need for measuring volumes in the hospital or clinic, which may introduce possible error and/or contamination, it is highly preferred that particular volumes of the neutral and acidic solutions be bottled so that adding the entire content of a container of the acidic solution to the entire content of a container of the neutral solution results in the correctly formulated tissue irrigating solution. The solutions may be mixed up to 24 hours before a surgical procedure without the occurrence of significant pH change, without the formation of detectable precipitates and without degradation. It is preferable, however, to prepare the irrigation solutions within 6 hours or less of surgery to avoid the possibility of microbial contamination of the irrigation solution.

Precautions must be taken to maintain sterility of the solutions and to insure correct mixing of the acidic and neutral solutions. While the manufacturer may take all due precautions to maintain quality control, carelessness by a technician may compromise the compositions. Any opening of a container, no matter how carefully performed, increases the likelihood of contamination in the contents. One method of substantially eliminating the possibility of improper mixing and reducing the likelihood of contamination would involve shipping the solutions in a container having a first chamber for the neutral solution, an isolated second chamber for the acidic solution and means to communicate the chambers without opening the container. The use of such containers are known for the shipment of multi-part medical solutions. For example, a container may have a lower chamber containing a measured volume of the neutral solution separated by a membrane from an upper chamber containing a measured volume of the acidic solution. The container cap may include a plunger means which, when depressed, causes a sharp point or blade depending therefrom to break the membrane. The container is thereafter agitated, as by shaking, to complete the sterile mixing in proper volume of the acidic and neutral solutions.

The proper mixing of the acidic and neutral solutions may also be carried out by aseptically removing the acidic solution from its package with a sterile syringe and needle and aseptically adding the acidic solution to the contents of the neutral solution package through the rubber stopper. Alternately, a sterile double-ended needle can be used to transfer the acidic solution to the neutral solution by aseptically inserting one end of the needle into the vial containing the acidic solution and then aseptically inserting the other end of the needle into the neutral solution package, whereby the vacuum that is maintained therein transfers the acidic solution to the neutral solution and is mixed.

The two-part irrigation solution system of the present invention also provides a safety advantage, should a technician fail to properly mix the two solutions. The larger volume neutral solution is physiologic so that there is less chance of toxicity if the neutral solution were used without the acidic solution being mixed therewith.

### EXAMPLE 1

The most preferred surgical irrigating solution of the present invention is prepared by the addition of the following Part II (acidic) solution to the Part I (neutral) solution:

A. Part I (Neutral Solution):

| Description | Concentration (w/v) |
|---|---|
| Sodium Chloride | 0.744% |
| Potassium Chloride | 0.0395% |
| Dibasic Sodium Phosphate (Anhydrous) | 0.0433% |
| Sodium Bicarbonate | 0.219%* |
| Hydrochloric Acid/Sodium Hydroxide | q.s. pH 7.4 |
| Water | q.s. |

*optionally, up to about an additional 20% excess may be added (i.e., a total sodium bicarbonate amount of up to about 0.270%)

B. Part II (Acidic Solution)

| Description | Concentration (w/v) |
|---|---|
| Compound X | 0.00146% |
| Polyoxyl-35 Castor Oil | 1.25% |
| Calcium Chloride (Dihydrate) | 0.385% |
| Magnesium Chloride (Hexahydrate) | 0.5% |
| Dextrose, Anhydrous | 2.3% |
| Ascorbic Acid | 0.002% |
| Sodium Ascorbate | 0.023% |
| Sodium Citrate | 0.059% |
| Hydrochloric Acid/Sodium Hydroxide | q.s. to pH 5.0 |
| Water | q.s. to 100% |

The Part II solution was prepared by the following procedure. A stock solution of Compound X in Cremophor EL® at 80 or 100 times the desired concentration was first prepared. Oxygen free water for injection (WFI) was then prepared. About 80% of the total water needed was then transferred to an aspirator bottle under a nitrogen purge. Calcium chloride, magnesium chloride, dextrose, sodium ascorbate, ascorbic acid and sodium citrate were then added to the aspirator bottle. The appropriate quantity of the Compound X stock solution was then added to the aspirator bottle solution. Following dissolution of all the components, the pH was adjusted and solution was brought to final volume. The solution was then nitrogen purged for approximately 8 to 10 hours and then sterile filtered, followed by the aseptically filling into sterile vials under a nitrogen head.

The surgical irrigating solution of this example is prepared by mixing 20 mL of Part II to 480 mL of Part I.

### EXAMPLE 2

Preferred acidic solutions (Part II) which may be used with a neutral solution of the present invention, e.g., the Part I composition of Example 1:

Part II (Acidic Solution)

| Description | Concentration (w/v) |
|---|---|
| Compound of formula (I) | 0.0025-0.007 |
| Polyoxyl-35 Castor Oil | 0.5 to 2.0% |
| Calcium Chloride (Dihydrate) | 0.36 to 1.8% |
| Magnesium Chloride (Hexahydrate) | 0.25 to 2.0% |
| Sodium Citrate | 0 to 0.2% |
| Dextrose, Anhydrous | 1.0 to 4.5% |
| Ascorbate Ion | 0.009 to 0.09% |
| Hydrochloric Acid/Sodium Hydroxide | q.s. to pH 4.0-7.0 |
| Water | q.s. |

The resultant surgical irrigating solution is prepared by mixing approximately 20 mL mL of Part II to approximately 480 mL of Part I.

### EXAMPLE 3

The following example demonstrates the stabilizing effect of ascorbate on a compound of formula (I) in an aqueous solution:

A concentrated stock solution of Compound X at approximately 100 times the desired concentration in Cremophor EL® was first prepared. An aliquot of the stock solution was diluted in water to yield a 25 μM concentration of Compound X in 1% w/v Cremophor El®. Ascorbic acid/sodium ascorbate was then added to the dilute Compound X solution to yield a final ascorbate ion concentration of 0.2% w/v. (A control solution was similarly prepared containing no ascorbate.) The aqueous solution was then sterile filtered into storage vials, and incubated at 40° for 1-11 weeks. At the appropriate time, an aliquot was taken and assayed for Compound X, using an HPLC with fluorometric detection. The results are reported in Table 1, as percent remaining of Compound X:

TABLE 1
Stabilization of Compound X with ascorbate in an aqueous solution

| | | Percent remaining of |
|---|---|---|
| Storage time | Percent remaining of | Compound X in |
| in weeks at | Compound X in water | water without |
| 40° C. | with ascorbate ion | ascorbate ion |
| Initial | 100 | 100 |
| 1 | 100 | 43 |
| 2 | 100 | 19 |
| 3 | 100 | 18 |
| 4 | 95 | 23 |
| 5 | — | — |
| 7 | — | N.D.* |
| 9 | 100 | — |
| 11 | 100 | N.D.* |

*Not Detectable

### EXAMPLE 4

The following example demonstrates the stability of the Part II solution of Example 1:

A Part II solution of Example 1, and an analogous solution containing no sodium ascorbate, ascorbic acid and citrate, were incubated at 40° C. for 1-26 weeks. The stability of Compound X was assayed at the respective time points using an HPLC method. The results are illustrated in Table 2, below.

TABLE 2
Comparison of the Stability of a Part II Solution of the Present
Invention with a solution without ascorbate

| Storage time | % remaining of | % remaning of Compound X |
|---|---|---|
| in weeks at | Compound X in the Part | in a Part II-like solution, |
| 40° C. | II solution of Example 1 | without ascorbate or citrate |
| Initial | 97 | 99 |
| 2 | — | 42 |
| 4 | 98 | 30 |
| 6 | — | 18 |
| 8 | 98 | 22 |
| 12 | 100 | — |
| 15 | 98 | — |
| 26 | 97 | — |

What is claimed is:

1. A two-part tissue irrigating system comprising:
(a) a sterile, neutral solution containing bicarbonate ions;
(b) a sterile, acidic solution containing dextrose, calcium ions, magnesium ions, a stabilizing amount of one or more physiological antioxidant(s) or derivative(s) thereof, and one or more compound(s) of formula (I):
A-X—(CH₂)ₙ—Y—(CH₂)ₘ—Z (I)
wherein:
A is an non-steroidal anti-inflammatory agent (NSAIA) originally having a carboxylic acid;
A-X is an ester or amide linkage derived from the carboxylic acid moiety of the NSAIA, wherein X is O or NR;
R is H, C₁-C₆ alkyl or C₃-C₆ cycloalkyl;
Y, if present, is O, NR, C(R)₂, CH(OH) or S(O)_{n′};
n is 2 to 4 and m is 1 to 4 when Y is O, NR, or S(O)_{n′};
n is 0 to 4 and m is 0 to 4 when Y is C(R)₂ or is not present;
n is 1 to 4 and m is 0 to 4 when Y is CH(OH);
n′ is 0 to 2; and
Z is: 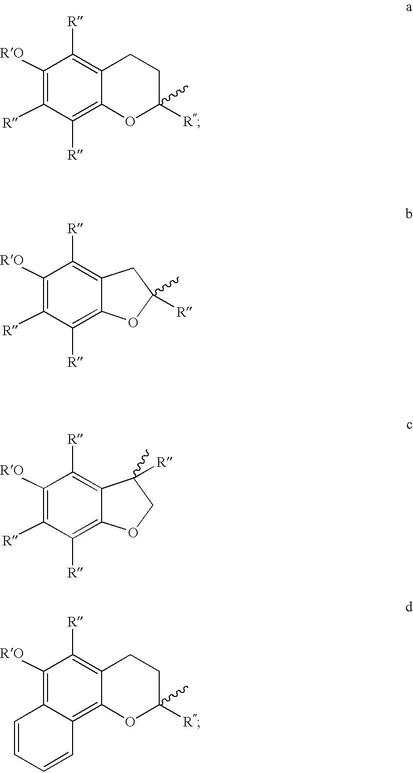
wherein:
R′ is H, C(O)R, C(O)N(R)₂, PO₃⁻, or SO₃⁻; and
R″ is H or C₁-C₆ alkyl; and pharmaceutically acceptable salts therefor;
wherein at least one of the solutions contains sodium ions, at least one of the solutions contains potassium ions and at least one of the solutions contains chloride ions; and
the acidic and neutral solutions when mixed together form the irrigating solution.

2. A system according to claim 1, wherein the neutral solution also contains phosphate ions and the physiological antioxidant is ascorbic acid/ascorbate.

3. A system according to claim 2, wherein the acidic solution further comprises one or more solubilizing agent(s).

4. A system according to claim 3, wherein the solubilizing agent in the acidic solution is polyoxyl-35 castor oil in an amount of between 0.5 and about 2.0% (w/v).

5. A system according claim 4, wherein the irrigating solution comprises between about:
130 and about 180 mM sodium ions;
3 and about 10 mM potassium ions;
1 and about 5 mM calcium ions;
0.5 and about 4 mM magnesium ions;
10 and about 50 mM bicarbonate ions;
2 and about 10 mM dextrose;
1 and about 5 mM phosphate ions;
0.02 and about 0.2 mM ascorbate ions,
0.2 and about 5 μM of a compound of formula (I);
0.02 and about 0.08% (w/v) of polyoxyl-35 castor oil;
0.0 and about 0.008% (w/v) of sodium citrate;
and the irrigating solution having a pH of between about 6.8 and about 8.0.

6. A system according to claim 5, wherein the volume ratio of the neutral solution to the acidic solution is between about 10:1 and about 40:1.

7. A system according to claim 6, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

8. A system according to claim 6, wherein the compound of formula (I) is selected from the group consisting of compounds wherein the non-steroidal anti-inflammatory agent is selected from the group consisting of: fenamic acids; indoles; and phenylalkanoic acids.

9. A system according to claim 6, wherein the compound of formula (I) is selected from the group consisting of compounds wherein the non-steroidal anti-inflammatory agent is selected from the group consisting of:
loxoprofen; tolfenamic acid; indoprofen; pirprofen; clidanac; fenoprofen; naproxen; fenclorac; meclofenamate, benoxaprofen; carprofen; isofezolac; aceloferac; fenbufen; etodolic acid; fleclozic acid; amfenac; efenamic acid; bromfenac; ketoprofen; fenclofenac; alcofenac; orpanoxin; zomopirac; diflunisal; flufenamic acid; niflumic acid; mefenamic acid; pranoprofen; zaltoprofen; indomethacin; sulindac; tolmetin; suprofen; ketorolac; flurbiprofen; ibuprofen; and diclofenac.

10. A system according to claim 9, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

11. A system according to claim 10, wherein the compound of formula (I) is selected from the group consisting of compounds wherein the non-steroidal anti-inflammatory agent is selected from the group consisting of naproxen, flurbiprofen and diclofenac.

12. A system according to claim 6, wherein the compound of formula (I) is selected from the group consisting of: 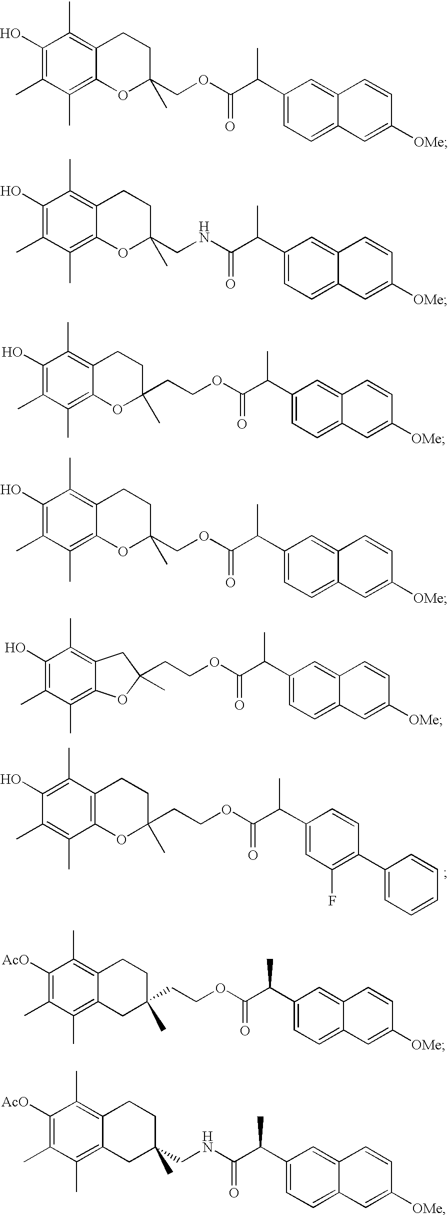
or stereoisomers thereof.

13. A system according to claim 1, wherein:
the neutral solution is comprised of:
0.744% (w/v) of sodium chloride;
0.0395% (w/v) potassium chloride;
0.0433% (w/v) dibasic sodium phosphate (anhydrous);
0.219 to 0.270% (w/v) sodium bicarbonate;
hydrochloric acid and/or sodium hydroxide; and
water;
the acidic solution comprised of:
0.00146% of a compound of formula (II): 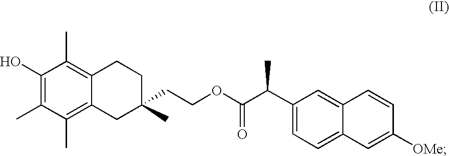
1.25% (w/v) polyoxyl-35 castor oil;
0.385% (w/v) calcium chloride (dihydrate);
0.5% (w/v) magnesium chloride (hexahydrate);
2.3% (w/v) dextrose, anhydrous;
0.002% (w/v) ascorbic acid;
0.023% (w/v) sodium ascorbate;
0.059% (w/v) sodium citrate;
hydrochloric acid and/or sodium hydroxide; and
water; and
wherein said neutral and acidic solutions are combined in a ratio of 24 to 1, respectively, to form the irrigating solution.

14. A two-part, tissue irrigating solution kit comprising:
(a) a package containing a sterile neutral solution containing bicarbonate ions, sodium ions and potassium ions, the package containing the solution under vacuum;
(b) a vial containing a sterile, acidic solution containing calcium ions, magnesium ions, dextrose, one or more physiological antioxidant(s) or derivative(s) thereof, and a compound of formula (I):
A-X—(CH₂)ₙ—Y—(CH₂)ₘ—Z (I)
wherein:
A is an non-steroidal anti-inflammatory agent (NSAIA) originally having a carboxylic acid;
A-X is an ester or amide linkage derived from the carboxylic acid moiety of the NSAIA, wherein X is O or NR;
R is H, C₁-C₆ alkyl or C₃-C₆ cycloalkyl;
Y, if present, is O, NR, C(R)₂, CH(OH) or S(O)_{n′};
n is 2 to 4 and m is 1 to 4 when Y is O, NR, or S(O)_{n′};
n is 0 to 4 and m is 0 to 4 when Y is C(R)₂ or is not present;
n is 1 to 4 and m is 0 to 4 when Y is CH(OH);
n′ is 0 to 2; and
Z is: 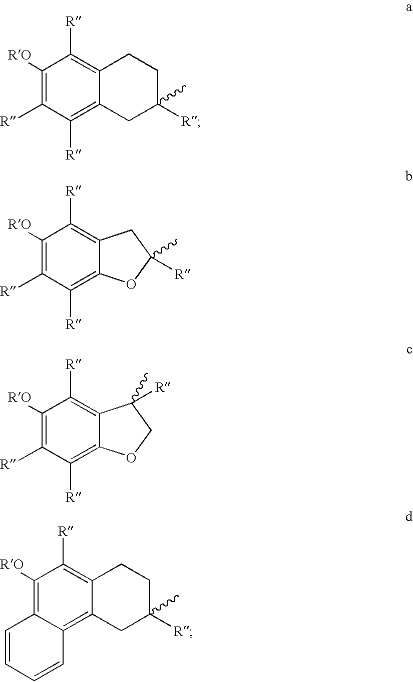
wherein:
R′ is H, C(O)R, C(O)N(R)₂, PO₃⁻, or SO₃⁻; and
R″ is H or C₁-C₆ alkyl; and pharmaceutically acceptable salts therefor;
the vial being closed with a rubber stopper;
(c) a sterile double-ended needle;
and at least one of said solutions containing chloride ions,
the acidic and neutral solutions being mixed together by aseptically inserting one end of the double-ended needle into the package containing the neutral solution, and the other end into the vial containing the acidic solution, and transferring the acidic solution into the neutral solution by the vacuum under which the neutral solution is maintained.

15. A kit according to claim 14, wherein the neutral solution also contains phosphate ions and the physiological antioxidant is ascorbic acid/ascorbate.

16. A kit according to claim 15, wherein the acidic solution further comprises a solubilizing agent.

17. A kit according to claim 16, wherein in the solubilizing agent is polyoxyl-35 castor oil in an amount of about 0.5 to 2.0% (w/v).

18. A kit according claim 17, wherein the irrigating solution comprises between about:
130 and about 180 mM sodium ions;
3 and about 10 mM potassium ions;
1 and about 5 mM calcium ions;
0.5 and about 4 mM magnesium ions;
10 and about 50 mM bicarbonate ions;
2 and about 10 mM dextrose;
1 and about 5 mM phosphate ions;
0.02 and about 0.2 mM ascorbate ions,
0.2 and about 5 μM of a compound of formula (I);
0.02 and about 0.08% (w/v) of polyoxyl-35 castor oil;
0.0 and about 0.008% (w/v) of sodium citrate;
and the irrigating solution having a pH of between about 6.8 and about 8.0.

19. A kit according to claim 18, wherein the volume ratio of the neutral solution to the acidic solution is between about 10:1 and about 40:1.

20. A kit according to claim 19, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

21. A kit according to claim 19, wherein the compound of formula (I) is selected from the group consisting of compounds wherein the non-steroidal anti-inflammatory agent is selected from the group consisting of: fenamic acids; indoles; and phenylalkanoic acids.

22. A kit according to claim 19, wherein the compound of formula (1) is selected from the group consisting of compounds wherein the non-steroidal anti-inflammatory agent is selected from the group consisting of:
loxoprofen; tolfenamic acid; indoprofen; pirprofen; clidanac; fenoprofen; naproxen; fenclorac; meclofenamate; benoxaprofen; carprofen; isofezolac; aceloferac; fenbufen; etodolic acid; fleclozic acid; amfenac; efenamic acid; bromfenac; ketoprofen; fenclofenac; alcofenac; orpanoxin; zomopirac; diflunisal; flufenamic acid; niflumic acid; mefenamic acid; pranoprofen; zaltoprofen; indomethacin; sulindac; tolmetin; suprofen; ketorolac; flurbiprofen; ibuprofen; and diclofenac.

23. A kit according to claim 22, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

24. A kit according to claim 23, wherein the compound of formula (I) is selected from the group consisting of compounds wherein the non-steroidal anti-inflammatory agent is selected from the group consisting of naproxen, flurbiprofen and diclofenac.

25. A kit according to claim 19, wherein the compound of formula (I) is selected from the group consisting of: 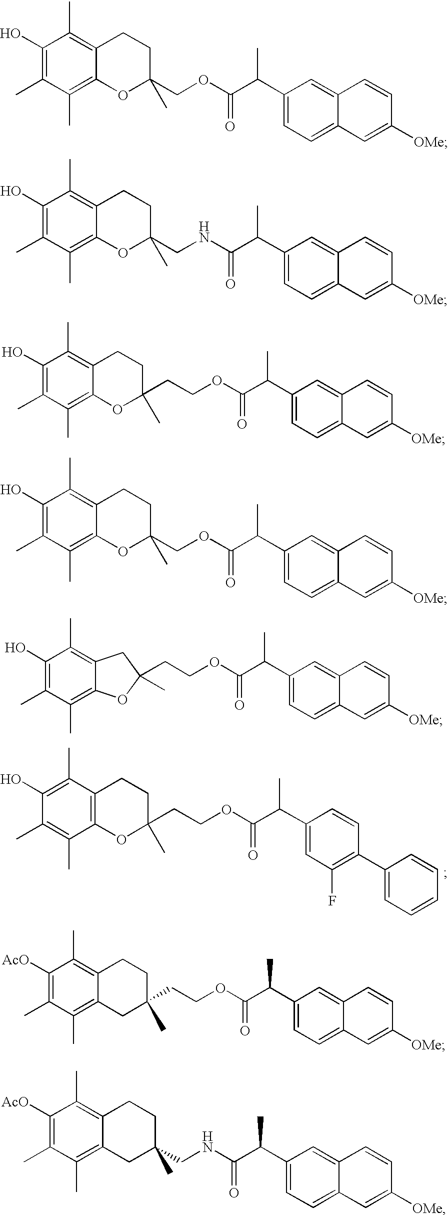
, or stereoisomers thereof.

26. A kit according to claim 14, wherein:
the neutral solution is comprised of:
0.744% (w/v) of sodium chloride;
0.0395% (w/v) potassium chloride;
0.0433% (w/v) dibasic sodium phosphate (anhydrous);
0.219 to 0.294% (w/v) sodium bicarbonate;
hydrochloric acid and/or sodium hydroxide; and
water;
the acidic solution comprised of:
0.00146% of a compound of formula (II): 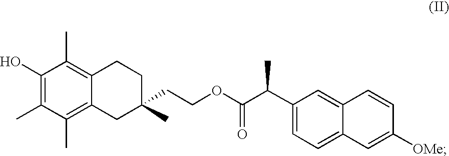
1.25% (w/v) polyoxyl-35 castor oil;
0.385% (w/v) calcium chloride (dihydrate);
0.5% (w/v) magnesium chloride (hexahydrate);
2.3% (w/v) dextrose, anhydrous;
0.002% (w/v) ascorbic acid;
0.023% (w/v) sodium ascorbate;
0.059% (w/v) sodium citrate;
hydrochloric acid and/or sodium hydroxide; and
water; and
wherein said neutral and acidic solutions are combined in a ratio of 24 to 1, respectively, to form the irrigating solution.

27. A method of treating mammalian tissues which comprises administering an irrigating solution to the mammilian tissues, the irrigation solution comprising:
(a) a sterile, neutral solution containing bicarbonate ions;
(b) a sterile, acidic solution containing dextrose, calcium ions, magnesium ions, a stabilizing amount of one or more physiological antioxidant(s) or derivatives thereof, and a therapeutically effective amount of one or more compound(s) of formula (I):
A-X—(CH₂)ₙ—Y—(CH₂)ₘ—Z (I)
wherein:
A is an non-steroidal anti-inflammatory agent (NSAIA) originally having a carboxylic acid;
A-X is an ester or amide linkage derived from the carboxylic acid moiety of the NSAIA, wherein X is O or NR;
R is H, C₁-C₆ alkyl or C₃-C₆ cycloalkyl;
Y, if present, is O, NR, C(R)₂, CH(OH) or S(O)_{n′};
n is 2 to 4 and m is 1 to 4 when Y is O, NR, or S(O)_{n′};
n is 0 to 4 and m is 0 to 4 when Y is C(R)₂ or is not present;
n is 1 to 4 and m is 0 to 4 when Y is CH(OH);
n′ is 0 to 2; and
Z is: 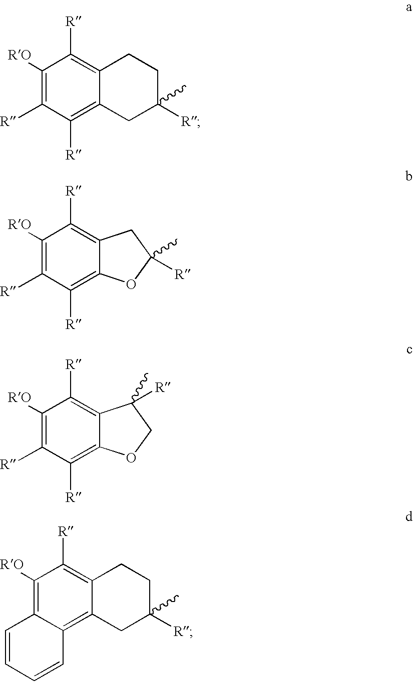
wherein:
R′ is H, C(O)R, C(O)N(R)₂, PO₃⁻, or SO₃⁻; and
R″ is H or C₁-C₆ alkyl; and pharmaceutically acceptable salts therefor;
wherein at least one of the solutions contains sodium ions, at least one of the solutions contains potassium ions and at least one of the solutions contains chloride ions; and
the acidic and neutral solutions when mixed together form the irrigating solution.

28. A method according to claim 27, wherein the neutral solution also contains phosphate ions and the physiological antioxidant is ascorbic acid/ascorbate.

29. A method according to claim 28, wherein the acidic solution further comprises a solubilizing agent.

30. A method according to claim 29, wherein the solubilizing agent is polyoxyl-35 castor oil in an amount of about 0.5-2.0% (w/v).

31. A method according claim 30, wherein the irrigating solution comprises between about:
130 and about 180 mM sodium ions;
3 and about 10 mM potassium ions;
1 and about 5 mM calcium ions;
0.5 and about 4 mM magnesium ions;
and about 50 mM bicarbonate ions;
2 and about 10 mM dextrose;
1 and about 5 mM phosphate ions;
0.02 and about 0.2 mM ascorbate ions,
0.2 and about 5 μM of a compound of formula (I);
0.02 and about 0.08% (w/v) of polyoxyl-35 castor oil;
0.0 and about 0.008% (w/v) sodium citrate;
and the irrigating solution having a pH of between about 6.8 and about 8.0.

32. An irrigating solution method according to claim 31, wherein the volume ratio of the neutral solution to the acidic solution is between about 10:1 and about 40:1.

33. A method according to claim 32, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d
R′ is H or C(O)CH₃; and
R″ is CH₃.

34. A method according to claim 32, wherein the compound of formula (I) is selected from the group consisting of compounds wherein the non-steroidal anti-inflammatory agent is selected from the group consisting of: fenamic acids; indoles; and phenylalkanoic acids.

35. A method according to claim 32, wherein the compound of formula (I) is selected from the group consisting of compounds wherein the non-steroidal anti-inflammatory agent is selected from the group consisting of:
loxoprofen; tolfenamic acid; indoprofen; pirprofen; clidanac; fenoprofen; naproxen; fenclorac; meclofenamate; benoxaprofen; carprofen; isofezolac; aceloferac; fenbufen; etodolic acid; fleclozic acid; amfenac; efenamic acid; bromfenac; ketoprofen; fenclofenac; alcofenac; orpanoxin; zomopirac; diflunisal; flufenamic acid; niflumic acid; mefenamic acid; pranoprofen; zaltoprofen; indomethacin; sulindac; tolmetin; suprofen; ketorolac; flurbiprofen; ibuprofen; and diclofenac.

36. A method according to claim 35, wherein the compound of formula (I) is selected from the group consisting of compounds wherein:
X is O or NR;
R is H or C₁ alkyl;
Y is CH(OH), and m is 0 to 2 and n is 1 or 2, or Y is not present, and m is 1 or 2 and n is 0 to 4;
Z is a, b or d;
R′ is H or C(O)CH₃; and
R″ is CH₃.

37. A method according to claim 36, wherein the compound of formula (I) is selected from the group consisting of compounds wherein the non-steroidal anti-inflammatory agent is selected from the group consisting of naproxen, flurbiprofen and diclofenac.

38. A method according to claim 32, wherein the compound of formula (I) is selected from the group consisting of: 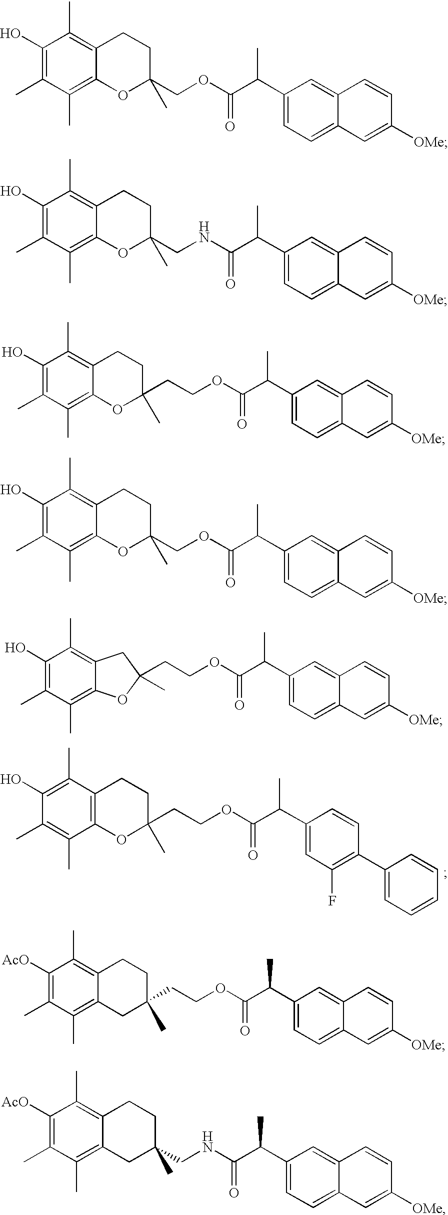
, or stereoisomers thereof.

39. A method according to claim 27, wherein:
the neutral solution is comprised of:
0.744% (w/v) of sodium chloride;
0.0395% (w/v) potassium chloride;
0.0433% (w/v) dibasic sodium phosphate (anhydrous);
0.219 to 0.294% (w/v) sodium bicarbonate;
hydrochloric acid and/or sodium hydroxide; and
water;
the acidic solution comprised of:
0.00146% of a compound of formula (II): 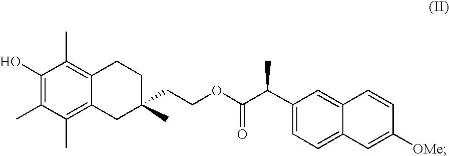
1.25% (w/v) polyoxyl-35 castor oil;
0.385% (w/v) calcium chloride (dihydrate);
0.5% (w/v) magnesium chloride (hexahydrate);
2.3% (w/v) dextrose, anhydrous;
0.002% (w/v) ascorbic acid;
0.023% (w/v) sodium ascorbate;
0.059% (w/v) sodium citrate;
hydrochloric acid and/or sodium hydroxide; and
water; and
wherein said neutral and acidic solutions are combined in a ratio of 24 to 1, respectively, to form the irrigating solution.

40. A method according to claim 27, wherein the irrigation solution is administered during refractive, cataract, glaucoma filtration or posterior segment surgery.

41. A method according to claim 39, wherein the irrigation solution is administered during refractive, cataract, glaucoma filtration or posterior segment surgery.
